(19) United States
(12) Patent Application Publication
(10) Pub. No.: US 2007/0025931 A1
(43) Pub. Date: Feb. 1, 2007

(54) Powdery preparations containing 2.4.6-trianilino-p-(carbo-2'-ethylhexyl-1'-oxi)-1.3.5-triazine

(75) Inventors: Werner Goedel, Ulm (DE); Birgit Blanz, Neustadt (DE); Valerie Andre, Ludwigshafen (DE); Helmut Auweter, Limburgerhof (DE); Michael Schaper, Ludwigshafen (DE); Andreas Habich, Speyer (DE); Thomas Wisniewski, Bensheim (DE)

Correspondence Address: CONNOLLY BOVE LODGE & HUTZ LLP, P.O. BOX 2207, WILMINGTON, DE, 19899-2207, US

(73) Assignee: BASF Aktiengesellschaft, Ludwigshafen (DE)

(21) Appl. No.: 10/571,769

(22) PCT Filed: Sep. 9, 2004
(86) PCT No.: PCT/EP04/10058

§ 371 Date: Mar. 14, 2006

(51) Int. Cl. A61K 8/49

(52) U.S. Cl. 424/59

(57) ABSTRACT

The invention relates to a method of producing powdered preparations comprising 2,4,6-trianilino-p-carbo-2′-ethylhexyl-1′-oxy)-1,3,5-triazine of formula I 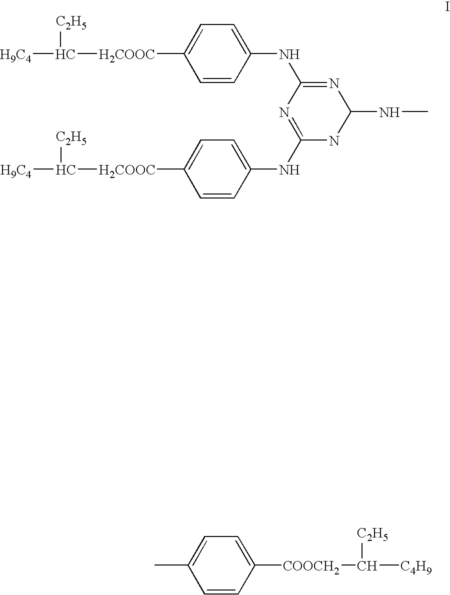 by the method comprising: a) dispersing the triazine of formula I in an aqueous molecularly disperse or colloidally disperse solution of a protective colloid and b) converting the dispersion obtained into a dry powder by removing the water and and drying, wherein the protective colloid used in process step a) is modified starch.

The invention relates to powdered preparations comprising 2,4,6-trianilino-p-(carbo-2′-ethylhexyl-1′-oxy)-1,3,5-triazine, to their preparation and to the use thereof as photostable photoprotective agents.

The quality and life span of many organic materials, for example plastics and coating materials, but also pharmaceutical and cosmetic preparations, can be adversely affected by the action of light, in particular by UV rays. These losses in quality frequently become evident in the case of plastics and coating materials from yellowing, discoloration, cracking or embrittlement of the material. In the case of pharmaceutical and cosmetic preparations, the effect of UV rays can lead to the degradation of the active ingredients present in the formulations.

The harmful effect of the ultraviolet part of solar radiation on the skin or hair, which in the widest sense are also an organic material, is likewise a problem which is increasing in importance. While rays having a wavelength of less than 290 nm (the UVC region) are absorbed by the ozone layer in the earth's atmosphere, rays in the range between 290 nm and 320 nm, the UVB region, cause an erythema, simple sunburn or even burns of varying severity on the skin.

A maximum for the erythema activity of sunlight is given as the relatively narrow range around 308 nm.

Numerous compounds are known for protecting against UVB radiation; these are, inter alia, triazine derivatives, derivatives of 3-benzylidenecamphor, of 4-aminobenzoic acid, of cinnamic acid, of salicylic acid, of benzophenone and of 2-phenylbenzimidazole.

It is also important to have available filter substances for the range between about 320 nm and about 400 nm, the UVA region, since its rays can cause reactions in cases of photosensitive skin. It has been proven that UVA radiation leads to damage of the elastic and collagenous fibers of the connective tissue, leading to premature aging of the skin, and that it is to be regarded as a cause of numerous phototoxic and photoallergic reactions. The harmful effect of UVB radiation can be intensified by UVA radiation.

To protect against UVA rays, derivatives of dibenzoylmethane are used, the photostability of which, however, is inadequate (Int. J. Cosm. Science 10, 53 (1988)).

However, UV radiation can also lead to photochemical reactions, in which case the photochemical reaction products then intervene in the skin's metabolism.

Such photochemical reaction products are mainly free-radical compounds, for example hydroxyl radicals. Undefined free-radical photo products formed in the skin itself can also trigger uncontrolled secondary reactions as a result of their high reactivity. However, singlet oxygen, a non-radical excited state of the oxygen molecule, can also arise during UV irradiation, as can short-lived epoxides and many others. Singlet oxygen, for example, differs from normal triplet oxygen (free-radical ground state) by virtue of its increased reactivity. However, activated, reactive (free-radical) triplet states of the oxygen molecule also exist.

Furthermore, UV radiation is a form of ionizing radiation. There is therefore the risk that ionic species will also form during UV exposure, which then for their part are able to intervene oxidatively in the biochemical processes.

One applications-relevant disadvantage of many UV filters is their poor solubility in water and/or in natural and synthetic oils, for example in silicone oils and in fatty acid triglycerides, as a result of which their use, for example in cosmetic formulations, is often restricted.

A further disadvantage associated with the application of some photoprotective agents is the appearance of skin irritations and allergies resulting from too high a skin permeability.

Numerous methods have already been published for improving the formulation properties of insoluble or only sparingly soluble UV absorbers.

For example, GB-A-2 303 549 describes a grinding process for the preparation of micronized insoluble organic UV absorbers in the presence of alkyl polyglycosides. The resulting micronizates can be incorporated into cosmetic photoprotective preparations.

GB-A-2 286 774 likewise describes a grinding process for the micronization of insoluble organic UV absorbers.

EP-A-1 127 567 describes aqueous dispersions of sparingly water-soluble or water-insoluble organic UV filter substances and dry powders produced therefrom, wherein they comprise at least one sparingly water-soluble or water-insoluble organic UV filter substance as colloidally disperse phase in amorphous of partially amorphous form. The use of the protective colloids specified in this specification—in particular gelatin or casein or caseinate—leads to powdered products whose solubility in cold water is unsatisfactory. In addition, gelatin and casein in cosmetic formulations can cause skin allergies.

It was then an object of the present invention to provide a method of producing triazine-containing photoprotective agent formulations which offer effective protection for organic material, in particular for the human skin and/or human hair, against UV rays, which are well tolerated by the skin and which can be incorporated easily both into lipophilic and also in particular into aqueous systems.

This object was achieved by a method of producing powdered preparations comprising 2,4,6-trianilino-p-(carbo-2′-ethylhexyl-1′-oxy)-1,3,5-triazine of the formula I 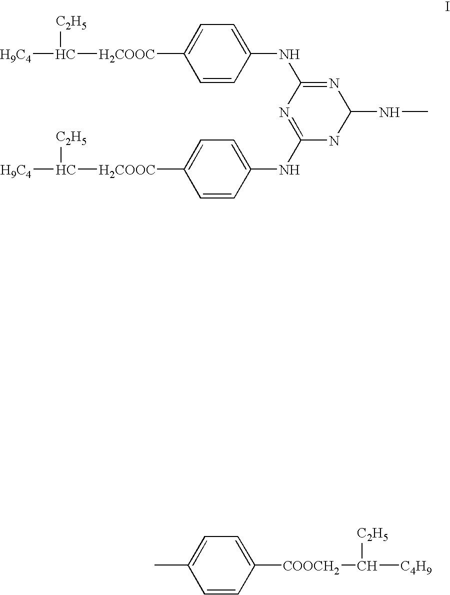
by
a) dispersing the triazine I in an aqueous molecularly disperse or colloidally disperse solution of a protective colloid and
b) converting the dispersion obtained into a dry powder by removing the water and, if appropriate, additionally used solvents, and drying,
   wherein the protective colloid used in process step a) is modified starch.

2,4,6-Trianilino-p-(carbo-2′-ethylhexyl-1′-oxy)-1,3,5-triazine of the formula I is marketed by BASF Aktiengesellschaft under the trade name Uvinul® T150 as a UVB filter. Uvinul® T150 is notable, inter alia, for good UV absorption properties with an exceptionally high absorbance coefficient>1500 at 314 nm.

For the purposes of the present invention, the term aqueous dispersions is understood as meaning both aqueous suspensions and emulsions. Preferred aqueous suspensions which may be mentioned are those in which the disperse phase comprises the triazine I as nanoparticulate particles.

For the purposes of the present invention, the term modified starch preferably comprises esters of starch with organic acids, e.g. with acetic acid and higher fatty acids (C₆-C₂₆), and with succinic acid, adipic acid and citric acid. The starch can be obtained here, inter alia, from corn, potatoes or wheat. A particularly preferred modified starch is octenyl succinate starch, which is marketed under the trade name HiCap® by National Starch or EmCap® by Cerestar.

A preferred variant of the method according to the invention is one in which the dispersion in stage a) comprises the following steps:
a₁) dissolving the triazine I in one or more water-miscible organic solvent(s) or in a mixture of water and one or more water-miscible organic solvent(s) or
a₂) dissolving the triazine I in one or more water-immiscible organic solvent(s) and
a₃) mixing the solution obtained after a₁) or a₂) with an aqueous molecularly disperse or colloidally disperse solution of modified starch, where the hydrophobic phase of the triazine I is formed as nanodisperse phase.

Depending on the type of solvent used, the nanodisperse phase in step a₃) may be solid nanoparticles [suspension; obtainable by combining a₁) and a₃)] or nanodroplets [emulsion; obtainable by combining a₂) and a₃)].

The water-miscible solvents used in stage a₁) are primarily water-miscible, thermally stable, volatile solvents comprising only carbon, hydrogen and oxygen, such as alcohols, ethers, esters, ketones and acetals. It is expedient to use those solvents which are at least 10% water-miscible, have a boiling point below 200° C., preferably below 100° C., and/or have fewer than 10 carbons. Particular preference is given to methanol, ethanol, n-propanol, isopropanol, 1,2-butanediol 1-methyl ether, 1,2-propanediol 1-n-propyl ether, tetrahydrofuran or acetone or mixtures thereof, and very particular preference is given to using isopropanol or acetone.

For the purposes of the present invention, the term “a water-immiscible organic solvent” is an organic solvent with a solubility in water at atmospheric pressure of less than 10%. Suitable possible solvents here are, inter alia, halogenated aliphatic hydrocarbons, such as, for example, methylene chloride, chloroform or carbon tetrachloride, carboxylic acid esters, such as diethyl carbonate, ethyl formate, methyl, ethyl or isopropyl acetate, and ethers, such as methyl tert butyl ether. Preferred water-immiscible organic solvents are the following compounds from the group consisting of dimethyl carbonate, propylene carbonate, ethyl formate, ethyl acetate, isopropyl acetate and methyl tert-butyl ether.

The dry powder in process step b) can be produced here, inter alia, by spray-drying, spray-cooling, freeze-drying, and by drying in a fluidized bed, convection drying or contact drying, it also being possible to carry out the drying in the presence of a coating material (powdering agent). Suitable coating agents are, inter alia, corn starch, silica and also tricalcium phosphate.

During the lyophilization of the nanoparticles according to the invention, cryoprotective substances such as, for example, trehalose or polyvinylpyrrolidones, can be added to the nanoparticles according to the invention.

Particular preference is given to an embodiment of the method according to the invention in which
a₁) the triazine I is dissolved in acetone or isopropanol or a mixture of water and acetone or water and isopropanol at temperatures in the range from 50 to 240° C.,
a₃) the solution obtained is mixed with an aqueous molecularly disperse or colloidally disperse solution of modified starch, in particular octenyl succinate starch, at temperatures in the range from 25 to 120° C. and
b) the suspension formed is spray-dried after removing the organic solvent.

The abovementioned dry powders are advantageously produced by dissolving the triazine of the formula I in acetone or isopropanol or a mixture of water and acetone or water and isopropanol at temperatures in the range from 50° C. to 240° C., in particular 100° C. to 200° C., particularly preferably in the range from 105° C. to 180° C.

To produce the molecularly disperse solution rapidly, the application of increased pressure, e.g. in the range from 20 bar to 80 200 bar, preferably 30 to 100 bar, may be advantageous.

The molecularly disperse solution obtained in this way is then mixed directly with the, if appropriate cooled, aqueous molecularly disperse or colloidally disperse solution of the modified starch, in particular octenyl succinate starch, in such a way that a mixing temperature of about 25° C. to 120° C., preferably 40° C. to 80° C., particularly preferably 45° C. to 70° C., is established.

In so doing, the solvent component is converted into the aqueous phase and the hydrophobic phase of the triazine is formed as nanodisperse phase.

The mixing in step a₃) can be carried out by initially introducing the solution comprising triazine, and metering in the aqueous solution of modified starch, or vice versa, or preferably by metering in both solutions simultaneously and continuously into a mixing chamber.

With regard to a more detailed description of the method and apparatus relating to the abovementioned dispersion, reference is made at this point to EP-B-0 065 193.

To increase the mechanical stability of the end product, in some cases it may be advantageous to add a further plasticizer to the colloid, such as sugars or sugar alcohols, e.g. sucrose, glucose, glucose syrup, dextrin, inverted sugar, sorbitol, mannitol or glycerol.

To increase the stability of the active ingredient against oxidative degradation, it may likewise be expedient to add stabilizers such as α-tocopherol, t-butylhydroxytoluene, t-butylhydroxyanisole, ascorbic acid or ethoxyquin. They can either be added to the aqueous phase or to the solvent phase, although they are preferably dissolved together with the triazine I in the solvent phase.

In addition, the photoprotective agent formulations can comprise low molecular weight surface-active compounds (emulsifiers) in a concentration of from 0.01 to 70% by weight, preferably 0.1 to 50% by weight, particularly preferably 0.5 to 20% by weight, based on the dry mass of the photoprotective agent formulation. Suitable as such are primarily amphiphilic compounds or mixtures of such compounds. In principle, all surfactants with an HLB value of from 5 to 20 are suitable. Suitable corresponding surface-active substances are, for example: esters of long-chain fatty acids with ascorbic acid, mono- and diglycerides of fatty acids and oxymethylation products thereof, esters of monofatty acid glycerides with acetic acid, citric acid, lactic acid or diacetyltartaric acid, polyglycerol fatty acid esters, such as, for example, the monostearate of triglycerol, sorbitan fatty acid esters, propylene glycol fatty acid esters and lecithin. Preference is given to using ascorbyl palmitate.

To increase the stability of the active ingredient against microbial degradation, it may be expedient to add preservatives to the preparation, such as, for example, methyl 4-hydroxybenzoate, propyl 4-hydroxybenzoate, sorbic acid or benzoic acid or salts thereof.

According to the invention, dry powders can thus be obtained which no longer lose their properties obtained in the primary dispersion. This means that the amorphous or partially crystalline character of the UV filter substance is retained. It is also a property according to the invention that these powders, upon redispersion, have the same particle size distribution which they had as primary dispersion with a deviation of 20%, preferably <15%.

A further preferred embodiment of the abovementioned method is one in which the suspension prepared in process step a) is ground before being converted into a dry powder.

The grinding method is preferably carried out by suspending the triazine I in crystalline form in an aqueous molecularly disperse or colloidally disperse solution of modified starch, and comminuting to the desired particle size by grinding.

The grinding can be carried out here in a manner known per se, e.g. using a ball mill. Depending on the type of mill used, grinding is carried out until the particles have an average particle size, determined via Fraunhofer diffraction, D[4.3] of from 0.01 to 100 μm, preferably from 0.02 to 50 μm, particularly preferably 0.05 to 20 μm, very particularly preferably 0.05 to 5 μm, in particular 0.1 to 1 μm. The term D[4.3] refers to the volume-weighted average diameter (see handbook for Malvern Mastersizer S, Malvern Instruments Ltd., UK).

By heating the aqueous suspension after the grinding process to a temperature above the melting point of the triazine I and then spray-drying the “melt emulsion”, it is possible to increase the amorphous fraction of the triazine in the resulting dry powder. Details regarding the grinding of active ingredients in aqueous protective colloid solutions are given in EP-B-0 498 824 and EP-B-0 684 973.

The invention also provides triazine-comprising powdered preparations obtainable by the abovementioned methods.

The novel photoprotective agent formulations are notable for the fact that they comprise the triazine of the formula 1, the amorphous fraction of which is in the range greater than 10%, preferably greater than 30%, particularly preferably in the range from 50 to 100%, very particularly preferably in the range from 75 to 99%. The degree of crystallinity of the triazine I can be determined here, for example, by X-ray diffraction measurements.

The content of triazine of the formula I in the photoprotective agent formulations according to the invention is in the range from 0.1 to 70% by weight, preferably in the range from 2 to 40% by weight, particularly preferably in the range from 3 to 30% by weight, very particularly preferably in the range from 5 to 25% by weight, based on the dry mass of the formulations.

The average particle size D[4.3] of the nanoparticulate particles in the aqueous dispersion is, depending on the formulation method, in the range from 0.01 to 100 μm, preferably in the range from 0.02 to 50 μm, particularly preferably in the range from 0.05 to 20 μm, very particularly preferably in the range from 0.05 to 5 μm, in particular 0.1 to 1 μm.

Whereas ground UV filter substances, when incorporated into skin creams, have an increased propensity for particle size growth, which can lead to a deterioration of the sun protection factor and to an unpleasant feel on the skin, the dry powders according to the invention do not have such tendencies on account of their matrix and protective colloid structure.

The formulations according to the invention—dispersions and dry powders prepared therefrom—are highly suitable for stabilizing organic material inter alia against the effect of light, oxygen and heat. They are added to the organic material to be stabilized in a concentration of from 0.01 to 10% by weight, preferably 0.01 to 5% by weight, particularly preferably from 0.02 to 2% by weight, based on the organic material, before, during or after its preparation.

Organic material is understood as meaning, for example, photographic recording materials, in particular photographic emulsions or precursors for plastics and surface coatings, but in particular plastics and surface coatings themselves.

Organic material, however, also means cosmetic preparations, such as, for example, creams, lotions, gels, lipsticks.

The present invention further relates to organic material stabilized against the action of light, oxygen and heat, in particular plastics and surface coatings, comprising 0.01 to 10% by weight, preferably 0.01 to 5% by weight, particularly preferably from 0.02 to 2% by weight, based on the total amount of the organic material, of the triazine I in the form of the formulations according to the invention.

For mixing the formulations according to the invention primarily with plastics, it is possible to use all known devices and methods for mixing stabilizing agents or other additives into polymers.

The organic material stabilized by the formulations according to the invention can, if appropriate, comprise further additives, e.g. antioxidants, light stabilizing agents, metal deactivators, antistatic agents, flame retardants, pigments and fillers.

Antioxidants and light stabilizers which can be added in addition to the formulations according to the invention are, for example, compounds based on sterically hindered phenols or costabilizers comprising sulfur or phosphorus.

Examples of such phenolic antioxidants are 2,6-di-tert-butyl-4-methylphenol, n-octadecyl-β-(3,5-di-tert-butyl-4-hydroxyphenyl)propionate, 1,1,3-tris(2-methyl-4-hydroxy-5-tert-butylphenyl)butane, 1,3,5-trimethyl-2,4,6-tris(3,5-di-tert-butyl-4-hydroxybenzyl)-benzene, 1,3,5-tris(3,5-di-tert-butyl-4-hydroxybenzyl)isocyanurate, 1,3,5-tris[β-(3,5-di-tert-butyl-4-hydroxyphenyl)propionylethyl]isocyanurate, 1,3,5-tris(2,6-dimethyl-3-hydroxy4-tert-butylbenzyl)isocyanurate and pentaerythritol tetrakis-[β-(3,5-di-tert-butyl-4-hydroxyphenyl)propionate].

Examples of suitable phosphorus-comprising antioxidants are tris(nonylphenyl)phosphite, distearylpentaerythritol diphosphite, tris(2,4-di-tert-butylphenyl)phosphite, tris(2-tert-butyl-4-methylphenyl)phosphite, bis(2,4-di-tert-butylphenyl)pentaerythritol diphosphite and tetrakis(2,4-di-tert-butylphenyl) 4,4′-biphenylenediphosphite.

Examples of sulfur-comprising antioxidants are dilauryl thiodipropionate, dimyristyl thiodipropionate, distearyl thiodipropionate, pentaerythritol tetrakis(β-laurylthiopropionate) and pentaerythritol tetrakis-(β-hexylthiopropionate).

Other antioxidants and light stabilizers which can be used together with the formulations according to the invention are, for example, 2-(2′-hydroxyphenyl)-benzotriazoles, 2-hydroxybenzophenones, aryl esters of hydroxybenzoic acids, α-cyanocinnamic acid derivatives, benzimidazolecarboxanilides, nickel compounds or oxalanilides.

Particularly good stabilization is achieved when at least one light stabilizer from the compound class of sterically hindered amines is also added in the usual concentration to the formulations according to the invention.

Examples of suitable sterically hindered amines are: bis(2,2,6,6-tetramethylpiperidyl)sebacate, bis(1,2,2,6,6-pentamethylpiperidyl)sebacate, the condensation product of 1-hydroxyethyl-2,2,6,6-tetramethyl-4-hydroxypiperidine and succinic acid, the condensation product of N,N′-di(2,2,6,6-tetramethylpiperidyl)hexamethylenediamine and 4-tert-octylamino-2,6-dichloro-1,3,5-triazine, tris(2,2,6,6-tetramethylpiperidyl)nitrilotriacetate, tetrakis(2,2,6,6-tetramethyl-4-piperidyl) 1,2,3,4-butanetetracarboxylate, 1,1′-(1,2-ethanediyl)-bis(3,3,5,5-tetramethylpiperazinone), the condensation products of 4-amino-2,2,6,6-tetramethylpiperidines and tetramethylolacetylenediureas.

Examples of plastics which can be stabilized by the compounds I according to the invention and may be mentioned are:
polymers of mono- and diolefins, such as, for example, low density or high density polyethylene, polypropylene, linear poly-1-butene, polyisoprene, polybutadiene, and copolymers of mono- or diolefins or mixtures of said polymers;
copolymers of mono- or diolefins with other vinyl monomers, such as, for example, ethylene/alkyl acrylate copolymers, ethylene/alkyl methacrylate copolymers, ethylene/vinyl acetate copolymers or ethylene/acrylic acid copolymers;
polystyrene and copolymers of styrene or -α-methylstyrene with dienes and/or acrylic derivatives, such as, for example, styrene/butadiene, styrene/acrylonitrile (SAN), styrene/ethyl methacrylate, styrene/butadiene/ethyl acrylate, styrene/acrylonitrile/methacrylate, acrylonitrile/butadiene/styrene (ABS) or methyl methacrylate/butadiene/styrene (MBS);
halogen-containing polymers, such as, for example, polyvinyl chloride, polyvinyl fluoride, polyvinylidene fluoride and copolymers thereof;
polymers derived from α,β-unsaturated acids and derivatives thereof, such as polyacrylates, polymethacrylates, polyacrylamides and polyacrylonitriles;
polymers derived from unsaturated alcohols and amines or acyl derivatives or acetals thereof, e.g. polyvinyl alcohol and polyvinyl acetate;
polyurethanes, polyamides, polyureas, polyesters, polycarbonates, polysulfonates, polyether sulfones and polyether ketones.

Furthermore, the formulations according to the invention can be used to stabilize aqueous emulsion paints and surface coatings, e.g. industrial finishes. Of these, particular attention is drawn to baking finishes, and, in turn, of these, automotive finishes, preferably two-coat finishes.

The formulations can be added in solid or liquid form to the surface coating. Their good solubility in surface coating systems is of particular advantage here.

Even in the case of the use as stabilizers in surface coatings, it is possible also to use the additional additives already listed, in particular antioxidants and light stabilizers.

The photoprotective agent formulations according to the invention are also very particularly preferably suitable as photostable UV filters in cosmetic and dermatological preparations for protecting human skin or human hair from solar rays and also from artificial light which has high UV contents, alone or together with compounds which absorb in the UV region and are known for cosmetic or pharmaceutical preparations. Thus, in the widest sense, the term organic materials also means human skin and human hair. The cosmetic and pharmaceutical preparations as such are of course also stabilized at the same time in order to remain effective for as long as possible.

Accordingly, the present invention also relates to cosmetic and pharmaceutical preparations comprising photoprotective agents for protecting human skin or human hair from UV light in the range from 280 to 400 nm, which comprise, as photostable UV filters and in a cosmetically or pharmaceutically suitable carrier, effective amounts of a formulation of the triazine I in amorphous or partially amorphous form—alone or together with compounds which absorb in the UV-A and UV-B region and are known per se for cosmetic and pharmaceutical preparations—the formulations being aqueous dispersions according to the invention mentioned in the introduction or the dry powders prepared therefrom.

The amount of triazine I in the form of the formulations according to the invention which is used in the cosmetic and pharmaceutical preparations is in the range from 0.05 to 20% by weight, preferably 0.1 to 10% by weight, particularly preferably in the range from 1 to 7% by weight, based on the total amount of the cosmetic and pharmaceutical preparation.

The cosmetic and pharmaceutical preparations comprising photoprotective agents are generally based on a carrier which comprises at least one oil phase. Preparations based solely on aqueous components are, however, also possible. Accordingly, suitable preparations are oils, oil-in-water and water-in-oil emulsions, creams and pastes, lip-protection stick compositions or grease-free gels.

Suitable emulsions are inter alia also O/W macroemulsions, O/W microemulsions or O/W/O emulsions containing triazine of the formula I present in dispersed form, the emulsions being obtainable by phase inversion technology, as in DE-A-197 26 121.

Customary cosmetic auxiliaries which may be suitable as additives are, for example, coemulsifiers, fats and waxes, stabilizers, thickeners, biogenic active ingredients, film formers, fragrances, dyes, pearlizing agents, preservatives, pigments, electrolytes (e.g. magnesium sulfate) and pH regulators. Suitable coemulsifiers are, preferably, known W/O and also O/W emulsifiers, such as, for example, polyglycerol esters, sorbitan esters or partially esterified glycerides. Typical examples of fats are glycerides; waxes which may be mentioned are inter alia beeswax, paraffin wax or microcrystalline waxes, if appropriate in combination with hydrophilic waxes. Stabilizers which may be used are metal salts of fatty acids, such as, for example, magnesium, aluminum and/or zinc stearate. Examples of suitable thickeners are crosslinked polyacrylic acids and derivatives thereof, polysaccharides, in particular xanthan gum, guar guar, agar agar, alginates and tyloses, carboxymethylcellulose and hydroxyethylcellulose, and also fatty alcohols, monoglycerides and fatty acids, polycrylates, polyvinyl alcohol and polyvinylpyrrolidone. The term biogenic active ingredients means, for example, plant extracts, protein hydrolyzates and vitamin complexes. Customary film formers are, for example, hydrocolloids, such as chitosan, microcrystalline chitosan or quaternary chitosan, polyvinylpyrrolidone, vinylpyrrolidone/vinyl acetate copolymers, polymers of the acrylic acid series, quaternary cellulose derivatives and similar compounds. Examples of suitable preservatives are formaldehyde solution, p-hydroxybenzoate or sorbic acid. Examples of suitable pearlizing agents are glycol distearic esters, such as ethylene glycol distearate, but also fatty acids and fatty acid monoglycol esters. Dyes which may be used are the substances suitable and approved for cosmetic purposes, as listed, for example, in the publication “Kosmetische Färbemittel” [Cosmetic Colorants] from the Farbstoffkommission der Deutschen Forschungsgemeinschaft [Dyes Commission of the German Research Council], published by Verlag Chemie, Weinheim, 1984. These dyes are usually used in a concentration of from 0.001 to 0.1% by weight, based on the total mixture.

An additional content of antioxidants is generally preferred. Thus, favorable antioxidants which can be used are all antioxidants which are suitable or customary for cosmetic and/or dermatological applications.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotene (e.g. β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thiorodoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximines, buthionine sulfones, penta-, hexa-, heptathionine sulfoximine) in very low tolerated doses (e.g. pmol to μmol/kg), also (metal) chelating agents (e.g. α-hydroxyfatty acids, palmitic acid, phytic acid, lactoferrin), α-hydroxy acids (e.g. citric acid, lactic acid, malic acid), humic acid, bile acid, bile extracts, bilirubin, biliverdin, EDTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives thereof (e.g. ascorbyl palmitate, Mg ascorbyl phosphate, ascorbyl acetate), tocopherol and derivatives (e.g. vitamin E acetate, tocotrienol), vitamin A and derivatives (vitamin A palmitate) and coniferyl benzoate of benzoin resin, rutic acid and derivatives thereof, α-glycosylrutin, ferulic acid, furfurylideneglucitol, carnosine, butylhydroxytoluene, butylhydroxyanisole, nordihydroguaiac resin acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide).

The amount of the abovementioned antioxidants (one or more compounds) in the preparations is preferably 0.001 to 30% by weight, particularly preferably 0.05 to 20% by weight, in particular 1 to 10% by weight, based on the total weight of the preparation.

If vitamin E and/or derivatives thereof are the antioxidant or antioxidants, it is advantageous to choose the respective concentration thereof from the range 0.001 to 10% by weight, based on the total weight of the formulation.

If vitamin A and/or derivatives thereof or carotenoids are the antioxidant or antioxidants, it is advantageous to choose the respective concentration thereof from the range 0.001 to 10% by weight, based on the total weight of the formulation.

Customary oil components in cosmetics are, for example, silicone oils, paraffin oil, glyceryl stearate, isopropyl myristate, diisopropyl adipate, cetylstearyl 2-ethylhexanoate, hydrogenated polyisobutene, vaseline, caprylic/capric triglycerides, microcrystalline wax, lanolin and stearic acid.

The total proportion of auxiliaries and additives can be 1 to 80% by weight, preferably 6 to 40% by weight, and the nonaqueous proportion (“active substance”) can be 20 to 80% by weight, preferably 30 to 70% by weight, based on the compositions. The compositions can be prepared in a manner known per se, i.e. for example by hot, cold, hot-hot/cold or PIT emulsification. This is a purely mechanical process, and no chemical reaction takes place.

Such sunscreen preparations can accordingly be in liquid, paste or solid form, for example as water-in-oil creams, oil-in-water creams and lotions, aerosol foam creams, gels, oils, marking pencils, powders, sprays or alcohol-aqueous lotions.

Finally, it is possible additionally to use further substances known per se which absorb in the UV region, provided they are stable in the overall system of the combination of UV filters to be used according to the invention.

The majority of photoprotective agents in the cosmetic and pharmaceutical preparations used to protect the human epidermis consists of compounds which absorb UV light in the UV-B region, i.e. in the range from 280 to 320 nm. For example, the proportion of the UV-A absorbers to be used according to the invention is 10 to 90% by weight, preferably 20 to 50% by weight, based on the total amount of UV-B and UV-A absorbing substances.

Suitable UV filter substances which are used in combination with the formulations to be used according to the invention are any UV-A and UV-B filter substances. Examples which may be mentioned are:

| | | CAS No. |
|---|---|---|
| No | Substance | (=acid) |
| 1 | 4-Aminobenzoic acid | 150-13-0 |
| 2 | 3-(4′-Trimethylammonium)benzylidenebornan-2-one | 52793-97-2 |
| | methylsulfate | |
| 3 | 3,3,5-Trimethylcyclohexyl salicylate(homosalate) | 118-56-9 |
| 4 | 2-Hydroxy-4-methoxybenzophenone(oxybenzone) | 131-57-7 |
| 5 | 2-Phenylbenzimidazole-5-sulfonic acid and its | 27503-81-7 |
| | potassium, sodium and triethanolamine salts | |
| 6 | 3,3′-(1,4-Phenylenedimethine)-bis(7,7-dimethyl- | 90457-82-2 |
| | 2-oxobicyclo[2.2.1]heptane-1-methanesulfonic | |
| | acid) and its salts | |
| 7 | Polyethoxyethyl 4-bis(polyethoxy)aminobenzoate | 113010-52-9 |
| 8 | 2-Ethylhexyl 4-dimethylaminobenzoate | 21245-02-3 |
| 9 | 2-Ethylhexyl salicylate | 118-60-5 |
| 10 | 2-Isoamyl 4-methoxycinnamate | 71617-10-2 |
| 11 | 2-Ethylhexyl 4-methoxycinnamate | 5466-77-3 |
| 12 | 2-Hydroxy-4-methoxybenzophenone-5-sulfonic acid | 4065-45-6 |
| | (sulisobenzone) and the sodium salt | |
| 13 | 3-(4′-Sulfobenzylidene)bornan-2-one and salts | 58030-58-6 |
| 14 | 3-Benzylidenebornan-2-one | 16087-24-8 |
| 15 | 1-(4′-Isopropylphenyl)-3-phenylpropane-1,3-dione | 63260-25-9 |
| 16 | 4-Isopropylbenzyl salicylate | 94134-93-7 |
| 17 | 3-Imidazol-4-ylacrylic acid and its ethyl ester | 104-98-3 |
| 18 | Ethyl 2-cyano-3,3-diphenylacrylate | 5232-99-5 |
| 19 | 2′-Ethylhexyl 2-cyano-3,3-diphenylacrylate | 6197-30-4 |
| 20 | Menthyl o-aminobenzoate or: | 134-09-8 |
| | 5-methyl-2-(1-methylethyl)-2-aminobenzoate | |
| 21 | Glyceryl p-aminobenzoate or: 1-glyceryl | 136-44-7 |
| | 4-aminobenzoate | |
| 22 | 2,2′-Dihydroxy-4-methoxybenzophenone | 131-53-3 |
| | (dioxybenzone) | |
| 23 | 2-Hydroxy-4-methoxy-4-methylbenzophenone | 1641-17-4 |
| | (mexenone) | |
| 24 | Triethanolamine salicylate | 2174-16-5 |
| 25 | Dimethoxyphenylglyoxalic acid or sodium | 4732-70-1 |
| | 3,4-dimethoxyphenylglyoxalate | |
| 26 | 3-(4′-Sulfobenzylidene)bornan-2-one and its salts | 56039-58-8 |
| 27 | 4-tert-Butyl-4′-methoxydibenzoylmethane | 70356-09-1 |
| 28 | 2,2′,4,4′-Tetrahydroxybenzophenone | 131-55-5 |
| 29 | 2,2′-Methylenebis[6(2H-benzotriazol-2-yl)-4- | 103597-45-1 |
| | (1,1,3,3-tetramethylbutyl)phenol] | |
| 30 | 2,2′-(1,4-Phenylene)-bis-1H-benzimidazole-4,6- | 180898-37-7 |
| | disulfonic acid, Na salt | |
| 31 | 2,4-bis[4-(2-Ethylhexyloxy)-2-hydroxy]phenyl-6- | 187393-00-6 |
| | (4-methoxyphenyl)-(1,3,5)-triazine | |
| 32 | 3-(4-Methylbenzylidene)camphor | 36861-47-9 |
| 33 | Polyethoxyethyl | 113010-52-9 |
| | 4-bis(polyethoxy)paraaminobenzoate | |
| 34 | 2,4-Dihydroxybenzophenone | 131-56-6 |
| 35 | 2,2′-Dihydroxy-4,4′-dimethoxybenzophenone-5,5′- | 3121-60-6 |
| | disodium sulfonate | |

Polymeric or polymer-bonded filter substances can also be used according to the invention.

The cosmetic and dermatological preparations according to the invention can additionally advantageously comprise inorganic pigments based on metal oxides and/or other metal compounds which are insoluble or sparingly soluble in water, for example the oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides. Particular preference is given to pigments based on TiO₂ and ZnO.

The inorganic pigments may be present here in hydrophobic form, i.e. surface-treated to repel water. This surface treatment may involve providing the pigments with a thin hydrophobic layer by a method known per se, as described in DE-A-33 14 742.

To protect human hair from UV rays, the photoprotective agent formulations according to the invention can be incorporated into shampoos, lotions, gels, hairsprays, hair colorants, aerosol foam creams or emulsions in concentrations of from 0.1 to 10% by weight, preferably 1 to 7% by weight. The respective formulations can inter alia be used for washing, coloring and for styling hair.

The formulations to be used according to the invention are usually notable for a particularly high absorbance in the UV-A radiation region with a sharp band structure. Moreover, they are readily soluble in cosmetic oils and can easily be incorporated into cosmetic formulations. The emulsions prepared with the formulations are particularly notable for their high stability, the formulations I themselves are notable for their high photostability, and the preparations prepared therewith are notable for their pleasant feel on the skin.

The UV filter action of the formulations according to the invention can also be utilized for stabilizing active ingredients and auxiliaries in cosmetic and pharmaceutical formulations.

The preparations according to the invention are notable for particularly high absorbance in the UV-B radiation region with a sharp band structure and high light protection factors.

In particular, the high sun protection factor of the preparations which was measured even at low concentrations of triazine compound I was surprising.

In addition, the preparations according to the invention have the advantage over other triazine-containing formulations of improved dispersibility in cold water.

The examples below serve to illustrate the present invention without limiting it.

### EXAMPLE 1

Preparation of a Uvinul® T150-Containing Dry Powder Having an Active Ingredient Content of 20% by Weight

a) Preparation of the Aqueous Dispersion

A molecularly disperse solution of 5 g of Uvinul® T150 in 95 g of acetone was passed at 150° C. to a mixing chamber, where it was mixed with 1300 g of a 30° C. hot aqueous solution of HiCap® (15 g/l). The entire process was carried out with a pressure limit of 40 bar in order to prevent evaporation of the solvent. After mixing, a colloidally disperse Uvinul® T150 dispersion with a white cloudy color was obtained.

Fraunhofer diffraction was used to determine the average volume distribution as D(4.3)=0.50 μm with a fines content of the distribution of 98.5%<1.22 μm.

b) Preparation of a Uvinul® T150-Containing Aqueous Dry Powder

Spray-drying the dispersion resulted in a dry powder having an active ingredient content of 20.56% by weight of Uvinul® T150 (content determination by means of UV/VIS spectroscopy). The dry powder could be redispersed in demineralized water again to form a white cloudy dispersion (hydrosol).

Fraunhofer diffraction was used to determine the average volume distribution in the redispersion as D(4.3)=0.65 μm with a fines content of the distribution of 97.1%<1.22 μm.

### EXAMPLE 2

Preparation of a Uvinul® T150-Containing Dry Powder Having an Active Ingredient Content of 20% by Weight

a) Preparation of the Aqueous Dispersion

A molecularly disperse solution of 10 g of Uvinul® T150 in 90 g of isopropanol/water (9:1, v/v) was passed at 150° C. to a mixing chamber, where it was mixed with 1000 g of a 30° C. hot aqueous solution of HiCap® (30 g/l). The entire process was carried out with a pressure limit of 40 bar in order to prevent evaporation of the solvent. After mixing, a colloidally disperse Uvinul® T150 dispersion with a white cloudy color was obtained.

Fraunhofer diffraction was used to determine the average volume distribution as D(4.3)=0.50 μm with a fines content of the distribution of 98.5%<1.22 μm.

b) Preparation of a Uvinul® T150-Containing Aqueous Dry Powder

Spray-drying the dispersion resulted in a dry powder having an active ingredient content of 20.56% by weight of Uvinul® T150 (content determination by means of UV/VIS spectroscopy). The dry powder could be redispersed in demineralized water again to form a white cloudy dispersion (hydrosol).

Fraunhofer diffraction was used to determine the average volume distribution in the redispersion as D(4.3)=0.65 μm with a fines content of the distribution of 97.1%<1.22 μm.

### EXAMPLE 3

Preparation of a Uvinul® T150-Containing Dry Powder Having an Active Ingredient Content of 15% by Weight

a) Preparation of the Aqueous Dispersion

25 g of Uvinul® T150 were dissolved in 216 g of isopropanol at room temperature to give a molecularly disperse solution. To precipitate out the Uvinul® T150 in colloidally disperse form, the solution was passed at 240° C. to a mixing chamber, where it was mixed with an aqueous solution of 45 g of HiCap in 1455 ml of demineralized water. The entire process was carried out with a pressure limit of 40 bar in order to prevent evaporation of the solvent. After mixing, a colloidally disperse Uvinul® T150 dispersion with a white cloudy color was obtained.

Fraunhofer diffraction was used to determine the average volume distribution as D(4.3)=0.52 μm with a fines content of the distribution of 98.5%<1.22 μm.

b) Preparation of a Uvinul® T150-Containing Aqueous Dry Powder

Spray-drying the dispersion resulted in a dry powder having an active ingredient content of 15.86% by weight of Uvinul® T150 (content determination by means of UV/VIS spectroscopy). The dry powder could be redispersed in demineralized water again to form a white cloudy dispersion (hydrosol).

Fraunhofer diffraction was used to determine the average volume distribution in the redispersion as D(4.3)=1.91 μm with a fines content of the distribution of 84.67%<1.22 μm.

Preparations

### EXAMPLE 4

Lip Care Composition

Mass content (% by weight)

| ad 100 | Eucerinum anhydricum |
|---|---|
| 10.00 | Glycerol |
| 10.00 | Titanium dioxide, micronized |
| 5.00 | Uvinul ® T150 dry powder from Example 1 |
| 8.00 | Octyl methoxycinnamate |
| 5.00 | Zinc oxide |
| 4.00 | Castor oil |
| 4.00 | Pentaerythritil stearate/caprate/caprylate adipate |
| 3.00 | Glyceryl stearate SE |
| 2.00 | Beeswax |
| 2.00 | Microcrystalline wax |
| 2.00 | Quaternium-18 bentonite |
| 1.50 | PEG-45/dodecyl glycol copolymer |

### EXAMPLE 5

Composition for Sunblock Containing Micropigments

Mass content (% by weight)

| ad 100 | Water |
|---|---|
| 10.00 | Octyl methoxycinnamate |
| 6.00 | PEG-7-Hydrogenated castor oil |
| 6.00 | Titanium dioxide, micronized |
| 5.00 | Uvinul ® T150 dry powder from Example 1 |
| 5.00 | Mineral oil |
| 5.00 | Isoamyl p-methoxycinnamate |
| 5.00 | Propylene glycol |
| 3.00 | Jojoba oil |
| 3.00 | 4-Methylbenzylidenecamphor |
| 2.00 | PEG-45/dodecyl glycol copolymer |
| 1.00 | Dimethicone |
| 0.50 | PEG-40 hydrogenated castor oil |
| 0.50 | Tocopheryl acetate |
| 0.50 | Phenoxyethanol |
| 0.20 | EDTA |

### EXAMPLE 6

Grease-Free Gel

Mass content (% by weight)

| ad 100 | Water |
|---|---|
| 8.00 | Octyl methoxycinnamate |
| 7.00 | Titanium dioxide, micronized |
| 5.00 | Uvinul ® T150 dry powder from Example 1 |
| 5.00 | Glycerol |
| 5.00 | PEG-25 PABA |
| 1.00 | 4-Methylbenzylidenecamphor |
| 0.40 | Acrylates C₁₀-C₃₀ alkyl acrylate crosspolymer |
| 0.30 | Imidazolidinylurea |
| 0.25 | Hydroxyethylcellulose |
| 0.25 | Sodium methylparaben |
| 0.20 | Disodium EDTA |
| 0.15 | Fragrance |
| 0.15 | Sodium propylparaben |
| 0.10 | Sodium hydroxide |

### EXAMPLE 7

Suncream (SPF 20)

Mass content (% by weight)

| ad 100 | Water |
|---|---|
| 8.00 | Octyl methoxycinnamate |
| 8.00 | Titanium dioxide, micronized |
| 6.00 | PEG-7-Hydrogenated castor oil |
| 5.00 | Uvinul ® T150 dry powder from Example 1 |
| 6.00 | Mineral oil |
| 5.00 | Isopropyl palmitate |
| 0.30 | Imidazolidinylurea |
| 3.00 | Jojoba oil |
| 2.00 | PEG-45/Dodecyl glycol copolymer |
| 1.00 | 4-Methylbenzylidenecamphor |
| 0.60 | Magnesium stearate |
| 0.50 | Tocopheryl acetate |
| 0.25 | Methylparaben |
| 0.20 | Disodium EDTA |
| 0.15 | Propylparaben |

### EXAMPLE 8

Water-Resistant Suncream

Mass content (% by weight)

| ad 100 | Water |
|---|---|
| 8.00 | Octyl methoxycinnamate |
| 5.00 | PEG-7-Hydrogenated castor oil |
| 5.00 | Propylene glycol |
| 4.00 | Isopropyl palmitate |
| 4.00 | Caprylic/capric triglyceride |
| 5.00 | Uvinul ® T150 dry powder from Example 1 |
| 4.00 | Glycerol |
| 3.00 | Jojoba oil |
| 2.00 | 4-Methylbenzylidenecamphor |
| 2.00 | Titanium dioxide, micronized |
| 1.50 | PEG-45/dodecyl glycol copolymer |
| 1.50 | Dimethicone |
| 0.70 | Magnesium sulfate |
| 0.50 | Magnesium stearate |
| 0.15 | Fragrance |

### EXAMPLE 9

Sun Milk (SPF 6)

Mass content (% by weight)

| ad 100 | Water |
|---|---|
| 10.00 | Mineral oil |
| 6.00 | PEG-7-Hydrogenated castor oil |
| 5.00 | Isopropyl palmitate |
| 3.50 | Octyl methoxycinnamate |
| 5.00 | Uvinul ® T150 dry powder from Example 1 |
| 3.00 | Caprylic/capric triglyceride |
| 3.00 | Jojoba oil |
| 2.00 | PEG-45/dodecyl glycol copolymer |
| 0.70 | Magnesium sulfate |
| 0.60 | Magnesium stearate |
| 0.50 | Tocopheryl acetate |
| 3.00 | Glycerol |
| 0.25 | Methylparaben |
| 0.15 | Propylparaben |
| 0.05 | Tocopherol |

### EXAMPLE 10

Day Lotion with UV Protection

Mass content (% by weight)

| | |
|---|---|
| ad 100 | Water |
| 2.00 | Cetearyl alcohol |
| 1.00 | Glycerol monostearate |
| 2.00 | Vaseline |
| 7.50 | Octyl methoxycinnamate |
| 4.00 | Octyl salicylate |
| 3.00 | Uvinul ® T150 dry powder from Example 1 |
| 1.50 | 4-tert-Butyl-4′-methoxydibenzoylmethane |
| 0.50 | Dimethicone |
| 5.00 | Propylene glycol |
| 0.20 | EDTA |
| 0.20 | Carbomer |
| 5.00 | C₁₂-C₁₅ Alkyl benzoate |
| 0.27 | Triethanolamine |
| 1.00 | Tocopheryl acetate |
| q.s. | Fragrance |

### EXAMPLE 11

Day Cream with UV Protection

Mass content (% by weight)

| | |
|---|---|
| ad 100 | Water |
| 2.00 | Cetearyl alcohol |
| 2.00 | Cetyl alcohol |
| 1.00 | Glycerol monostearate |
| 2.00 | Vaseline |
| 7.50 | Octyl methoxycinnamate |
| 4.00 | Octyl salicylate |
| 3.00 | Uvinul ® T150 dry powder from Example 1 |
| 1.50 | 4-tert-Butyl-4′-methoxydibenzoylmethane |
| 4.00 | Propylene glycol |
| 0.20 | EDTA |
| 0.20 | Carbomer |
| 0.20 | Xanthan |
| 0.20 | C₁₀-C₃₀ Alkyl acrylate crosspolymer |
| 5.00 | C₁₂-C₁₅ Alkyl benzoate |
| 0.54 | Triethanolamine |
| 1.00 | Tocopheryl acetate |
| q.s. | Fragrance |
| q.s. | Preservative |

### EXAMPLE 12

Liquid Make Up

Mass content (% by weight)

| | |
|---|---|
| ad 100 | Water |
| 2.00 | Cetearyl alcohol |
| 2.00 | Ceteareth 25 |
| 6.00 | Glycerol monostearate |
| 1.00 | Cetyl alcohol |
| 8.00 | Paraffin oil |
| 7.00 | Cetearyl octanoate |
| 0.2 | Dimethicone |
| 3.00 | Propylene glycol |
| 1.00 | Panthenol |
| 3.00 | Uvinul ® T150 dry powder from Example 1 |
| 1.50 | 4-tert-Butyl-4′-methoxydibenzoylmethane |
| 3.50 | Octyl methoxycinnamate |
| 0.1 | Bisabolol |
| 5.70 | Titanium dioxide |
| 1.10 | Iron oxide |
| q.s. | Fragrance |

### EXAMPLE 13

Hair Gel with Sun Protection

Mass content (% by weight)

| | |
|---|---|
| ad 100 | Water |
| 1.20 | Carbomer |
| 0.50 | Hydroxyethylcellulose |
| 4.00 | Triethanolamine |
| 0.70 | PEG-40 Hydrogenated castor oil |
| 1.50 | Uvinul ® T150 dry powder from Example 1 |
| 0.70 | 4-tert-Butyl-4′-methoxydibenzoylmethane |
| 2.80 | Octyl methoxycinnamate |
| 5.00 | Propylene glycol |
| 0.01 | EDTA |
| q.s. | Fragrance |
| q.s. | Sicovit Patent Blue 85 E 131 |

1. A method of producing a powdered preparation comprising 2,4,6-trianilino-p-carbo-2′-ethylhexyl-1′-oxy)-1,3,5-triazine of formula I 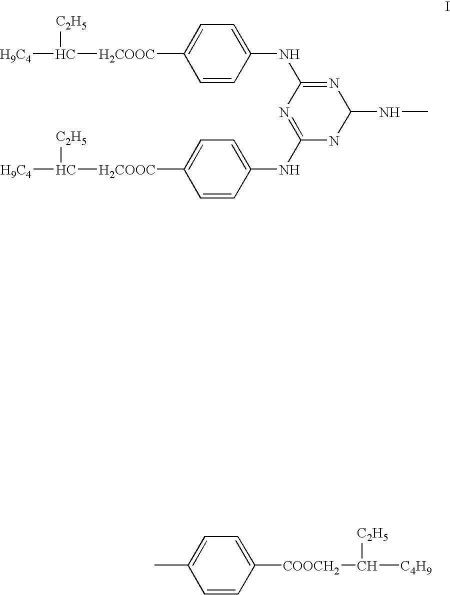
the method comprising:
a) dispersing the triazine of formula I in an aqueous molecularly disperse or colloidally disperse solution of a protective colloid; and
b) converting the dispersion obtained into a dry powder by removing the water and drying,
wherein the protective colloid used in process step a) is modified starch.

2. The method according to claim 1, wherein the dispersion in stage a) comprises the following steps:
a₁) dissolving the triazine of formula I in one or more water-miscible organic solvent(s) or in a mixture of water and one or more water-miscible organic solvent(s); or
a2) dissolving the triazine of formula I in one or more water-immiscible organic solvent(s); and
a₃) mixing the solution obtained after a₁) or a₂) with an aqueous molecularly disperse or colloidally disperse solution of modified starch, where the hydrophobic phase of the triazine of formula I is formed as nanodisperse phase.

3. The method according to claim 1, wherein the drying in process step b) is conducted in the presence of a coating material.

4. The method according to claim 1, wherein the dispersing of the triazine of formula I in an aqueous molecularly disperse or colloidally disperse solution includes a preparation of a suspension with the modified starch.

5. The method according to claim 4, further comprising grinding the suspension prior to conversion into a dry powder.

6. The method according to claim 4, wherein
a₁) the triazine of formula I is dissolved in acetone or isopropanol a mixture of water and acetone, or water and isopropanol at temperatures from 50 to 240° C.,
a₃) the solution obtained is mixed with an aqueous molecularly disperse or colloidally disperse solution of modified starch at temperatures from 25 to 120° C. and
b) the suspension formed is spray-dried after removing the organic solvent.

7. The method according to claim 1, wherein the dispersing of the triazine of formula I in an aqueous molecularly disperse or colloidally disperse solution includes a preparation of an emulsion with the modified starch.

8. The method according to claim 1, wherein the protective colloid used is octenyl succinate starch.

9. A triazine-containing powdered preparation obtainable by a method according to claim 1.

10. The preparation according to claim 9 with a content of triazine of formula I of from 0.1 to 70% by weight.

11. The use of the triazine-containing powder defined according to claim 9 as a photostable UV filter in cosmetic and dermatological preparations.

12. The method according to claim 1, wherein the molecularly disperse or colloidally disperse solution includes one or more organic solvents, which are removed prior to the conversion to the dry powder.

13. The method according to claim 4, wherein the protective colloid used is octenyl succinate starch.

14. The method according to claim 7, wherein the protective colloid used is octenyl succinate starch.

15. A powdered preparation containing diethylamino hydroxybenzoyl hexyl benzoate, obtainable by a method according to claim 4.

16. A powdered preparation containing diethylamino hydroxybenzoyl hexyl benzoate, obtainable by a method according to claim 7.

17. A powdered preparation comprising diethylamino hydroxybenzoyl hexyl benzoate of formula I 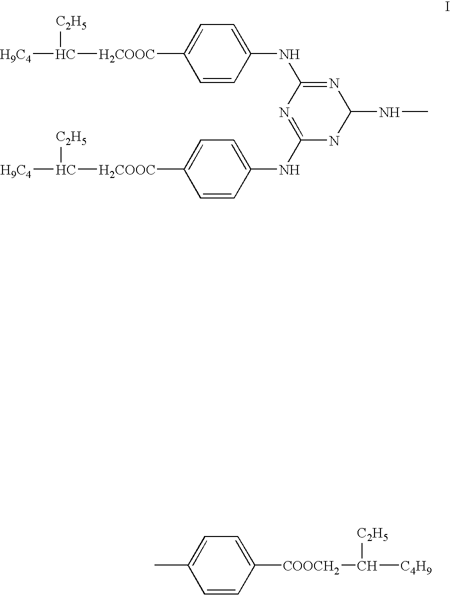
the preparation prepared by a process comprising:
a) dispersing the benzoyl benzoate of formula I in an aqueous molecularly disperse or colloidally disperse solution of a protective colloid; and
b) converting the dispersion obtained into a dry powder by removing the water and drying,
wherein the powdered preparation is used as a photostable UV filter in a dermatological preparation.

18. The preparation according to claim 17, wherein the powdered preparation is present from 5% to 25% by weight in the dermatological preparation, based on the dry weight of the dermatological preparation.

19. The method according to claim 2, wherein the average particle size of the nanodisperse phase is from 0.05 μm to 20 μm.

20. The preparation according to claim 18, wherein the molecularly disperse or colloidally disperse solution includes a hydrophobic phase of the benzoyl benzoate of formula I as a nanodisperse phase, wherein the average particle size of the nanodisperse phase is from 0.05 μm to 20 μm.
